(19) United States
(12) Patent Application Publication
(10) Pub. No.: US 2008/0097364 A1
(43) Pub. Date: Apr. 24, 2008

(54) METHOD OF MANUFACTURING A SANITARY NAPKIN COLOR-PACKAGED

(76) Inventor: Yinhai YANG, Tian Jin (CN)

Correspondence Address: WANG & HO, 66 HILLTOP ROAD, MILLINGTON, NJ, 07946, US

(21) Appl. No.: 11/551,717

(22) Filed: Oct. 22, 2006

(51) Int. Cl. A61F 13/15

(52) U.S. Cl. 604/385.02

(57) ABSTRACT

A method of manufacturing a sanitary napkin or sanitary pad color-packaged, comprising the steps: (1) producing a number of finished sanitary articles; (2) packaging the finished articles with a warp material printed with non-uniformly distributed graphic design elements varying in color and shape to produce a number of inner package showing different graphic design elements; and (3) packaging a number (for example 10) of the inner packages having varying graphic design elements on the wrap with a transparent wrap material to product a larger package convenient for transportation and sale.

### CROSS REFERENCE TO RELATED APPLICATIONS

This application is a CIP application of PCT application No. PCT/CN2005/000189, filed Feb. 16, 2005, which claims priority to Chinese Patent Application No. 200410019074.6, filed Apr. 23, 2004. The contents of the two prior applications are hereby incorporated by reference.

### FIELD OF THE INVENTION

The present invention relates to a method of manufacturing sanitary articles, particularly relates to a method of manufacturing feminine sanitary articles using a novel internal color-packaging technique.

### BACKGROUND OF THE INVENTION

In recent years transparent outer packaging has become popular in the industry. Going with the trend, more and more sanitary articles have been packaged with a transparent outer package. However, inside the transparent outer package, the individual article is still internally packaged using a single color.

Similarly, sanitary articles for women and children have also adopted the transparent outer packaging method and, due to the see-through nature of the outer package, the design of the inner packages for individual articles becomes the focus of consumers' attention. The single color inner packaging seems too monotonous to consumers and unable to appeal to their usual psychological need for varieties. Therefore, some manufacturers have begun to use multi-color packaging for individual inner packages, that is, inside the transparent outer package articles are individually packaged with wraps of different colors. The prior art method for producing multi-color inner packages is as follows: firstly, a certain amount of individual articles (for example, sanitary napkins or sanitary pads) are individually packaged with a wrap of a single color, such as yellow, blue, etc, and stored in a temporary box and then this procedure is repeated for another color; secondly, individual articles in different color inner packages are manually taken from the temporary boxes and mixed according to a predetermined ratio; lastly, a certain number of articles properly mixed in terms of the color of their inner packages are wrapped within a transparent outer package (usually 10 inner packages for one outer package). Because of the need for changing wraps of different colors from time to time, the packaging process becomes complex and discontinuous. At the same time, with manually sorting and mixing inner packages of different colors, it is very labor-intensive and the use of a large number of the temporary boxes occupies valuable space in the workplace. Thus, manually sorting and mixing inner packages is slow and ineffective. Furthermore, because of involvement of human handling of the inner packages, there is a potential issue of secondary contamination of the sanitary articles during the manufacturing process. All these together create serious problems for package process management, quality insurance and production economy.

### SUMMARY OF THE INVENTION

Accordingly, it is an object of the present invention to overcome the drawbacks in the prior art, and to provide a simple, efficient and relatively low-cost method of manufacturing and packaging sanitary articles with varied colorful inner packages. This method is applicable to sanitary products including, but not limited to, baby diaper, pantyliner, sanitary napkin, and sanitary pad. It is achieved with the technique scheme described as follows:

A method of manufacturing a sanitary product comprises the steps of: (a) producing a plurality of individual sanitary articles, which comprise an absorbent layer, sandwiched between a top layer and a bottom layer; (b) packaging sanitary articles individually with a piece of inner packaging wrap material on which there are printed with non-uniformly distributed different graphic design units so that the sanitary articles packaged with the same piece of inner packaging wrap material may each have a different set or pattern of the graphic design units shown on their inner package; and (c) packaging a number of inner packaged units with a piece of transparent outer wrap to provide a larger package convenient for transportation and sale.

In one embodiment of the present invention, the inner wrap material is printed with a plurality of color stripes, arranged to form one or more geometric shapes, such as, for example, square, triangle or rectangle (collectively referred to as graphic design unit). The graphic design unit is of the same or different length or width. The repeating units may be separated with the same or different distances/intervals. The width of the stripes is at least 10 mm. The stripes may be printed alternately in at least two colors of any kind, including red, yellow, blue, green, orange, black, white, etc. The wrap material can have a color background, for example, blue, yellow, green, pink, purple, etc.

In another embodiment of the present invention, the inner wrap material is printed with a plurality of color graphic images, which may be simple geometrical shapes, outlines of common objects, or shapes of plants or animals. The geometrical shapes can be rectangle, trap, triangle or wave pattern, etc., and can be arranged randomly, either being separated from each other or overlapping with each. The graphic images may be printed in any number of ways on the inner wrap material as long as the inner packages produced from the same inner wrap material can have different graphic patterns or images (collectively referred to as graphic design units). The graphic design unit can be outlines or shapes of any objects, plants, animals, English character, Chinese character or any random graphic patterns synthesized using computer.

Compared with the prior art method, the packaging method of the present invention is simplified. However, it achieves an unexpected effect in spite of its simplicity—produces a number of inner packages each having a varied set of graphic design units (color, shape, images) from a single inner wrap material. With this method, the inner packages are directly packaged with an outer wrap without manually sorting or mixing different inner packages as an intermediate step. The production and packaging are continuous and efficient. It can increase productivity by 75% and alleviate the labor intensity and secondary contamination problems.

The various features of novelty which characterize the invention are pointed out with particularity in the claims annexed to and forming a part of this disclosure. For a better understanding of the invention, its operating advantages, and specific objects attained by its use, reference should be made to the drawings and the following description in which there are illustrated and described preferred embodiments of the invention.

### DETAILED DESCRIPTION OF PARTICULAR EMBODIMENTS

A detailed description of the present invention is provided hereinafter with reference to the specific examples.

### EXAMPLE I

Paper sheets (free of dusts) and filer core was compounded with non-woven fabric to make a sanitary napkin or sanitary pad using a conventional method. The sanitary napkin or sanitary pad was packaged individually with an inner wrap material (coated with silica gel), which can be of any suitable sheet materials usually and conventionally used in the industry. However, the inner wrap material was printed with squares and rectangles made up with equal distanced stripe units in any 4 different colors, which were 40 mm in width. The individually packaged sanitary napkins or sanitary pads were further packaged with a transparent outer wrap material into a larger package. Each outer package contained 10 individual inner packages of varied color and/or image patterns, which were visible because the outer package was transparent. The outer packages were then press packed, boxed and warehoused, ready for transportation and sale.

### EXAMPLE 2

Paper sheets (free of dusts) and filer core was compounded with non-woven fabric to make a sanitary napkin or sanitary pad using a conventional existing method. The sanitary napkin or sanitary pad was packaged individually with an inner wrap material (not coated with silica gel), which can be of any suitable sheet materials usually and conventionally used in the industry. However, the inner wrap material was printed with rectangles, traps, triangles or wave patterns made up of non-equal distanced stripe units in any 6 different colors, which were 50 mm in width. The individually packaged sanitary napkins or sanitary pads were further packaged with a transparent outer wrap material into a larger package, containing 8 individual inner packages of varied color and/or image patterns, which are visible because the outer package is transparent. The products were then press packed, boxed and warehoused, ready for transportation and sale.

### EXAMPLE 3

Paper sheets (free of dusts) and filer core was compounded with non-woven fabric to make a sanitary napkin or sanitary pad using a conventional existing method. The sanitary napkin or sanitary pad was packaged individually with an inner wrap material (not coated with silica gel), which can be of any suitable sheet materials usually and conventionally used in the industry. However, the inner wrap material was printed with rectangles, traps, triangles or wave patterns made up of non-equal distanced stripe units in any 4 different colors, which were 20 mm in width. The individually packaged sanitary napkins or sanitary pads were further packaged with a transparent outer wrap material into a larger package, containing 6 individual inner packages of varied color and/or image patterns, which are visible because the outer package is transparent. The products were then press packed, boxed and warehoused, ready for transportation and sale.

### EXAMPLE 4

Paper sheets (free of dusts) and filer core was compounded with non-woven fabric to make a sanitary napkin or sanitary pad using a conventional existing method. The sanitary napkin or sanitary pad was packaged individually with an inner wrap material (coated with silica gel), which can be of any suitable sheet materials usually and conventionally used in the industry. However, the inner wrap material was printed with squares and rectangles made up with equal distanced stripe units in any 5 different colors, which were 60 mm in width. The individually packaged sanitary napkins or sanitary pads were further packaged with a transparent outer wrap material into a larger package, containing 10 individual inner packages of varied color and/or image patterns, which were visible because the outer package was transparent. The products were then press packed, boxed and warehoused, ready for transportation and sale.

### EXAMPLE 5

Paper sheets (free of dusts) and filer core was compounded with non-woven fabric to make a sanitary napkin or sanitary pad using a conventional existing method. The sanitary napkin or sanitary pad was packaged individually with an inner wrap material (coated with silica gel), which can be of any suitable sheet materials usually and conventionally used in the industry. However, the inner wrap material was printed with rectangles, traps, triangles or wave patterns made up of non-equal distanced stripe units in any 2 different colors, which were 40 mm in width. The individually packaged sanitary napkins or sanitary pads were further packaged with a transparent outer wrap material into a larger package, containing 10 individual inner packages of varied color and/or image patterns, which are visible because the outer package is transparent. The products were then press packed, boxed and warehoused, ready for transportation and sale.

### EXAMPLE 6

Paper sheets (free of dusts) and filer core was compounded with non-woven fabric to make a sanitary napkin or sanitary pad using a conventional existing method. The sanitary napkin or sanitary pad was packaged individually with an inner wrap material (coated with silica gel), which can be of any suitable sheet materials usually and conventionally used in the industry. However, the inner wrap material are printed with various images such as flowers, animals, English characters, Chinese characters and random graphic patterns synthesized by a computer. The images may be printed on a background color such as blue, yellow, green, pink, purple, etc. The individually packaged sanitary napkins or sanitary pads were further packaged with a transparent outer wrap material into a larger package, containing 10 individual inner packages of varied color and/or image patterns, which were visible because the outer package was transparent. The products were then press packed, boxed and warehoused, ready for transportation and sale.

While there have been described and pointed out fundamental novel features of the invention as applied to a preferred embodiment thereof, it will be understood that various omissions and substitutions and changes, in the form and details of the embodiments illustrated, may be made by those skilled in the art without departing from the spirit of the invention. The invention is not limited by the embodiments described above which are presented as examples only but can be modified in various ways within the scope of protection defined by the appended patent claims.

What is claimed is:

1. A method of manufacturing sanitary articles, comprising the steps of following: (a) processing an absorbent layer, a top layer and a bottom layer to a finished sanitary article; (b) packaging a plurality of said sanitary articles with a piece of wrap material to produce inner packages each containing a single said sanitary article, where said wrap material is printed with a plurality of graphic design units, which are non-uniformly distributed on said wrap material so that at least some of said inner packages bear a different set of said graphic design units; and (c) packaging a number of said inner packages with a piece of outer wrap material to afford a larger package convenient for transportation and sale.

2. The method of claim 1, wherein said outer wrap material is a see-through material.

3. The method of claim 1, wherein said graphic design units are each composed of a plurality of stripes of different colors, are of same or different sizes, and are repeated at same or different intervals.

4. The method of claim 1, wherein said graphic design units comprise one or more kinds of geometric shapes.

5. The method of claim 1, wherein said graphic design units comprise one or more outlines or images of plants.

6. The method of claim 1, wherein said graphic design units comprise one or more outlines or images of animals.

7. The method of claim 1, wherein said graphic design units comprise one or more alphabets or English words.

8. The method of claim 1, wherein said graphic design units comprise one or more Chinese characters.

9. The method of claim 1, wherein said graphic design units comprise one or more symbols generated by computer.

10. The method of claim 1, wherein said graphic design units are printed with at least two different colors selected from the group consisting of red, blue, yellow, green, pink, purple, white and black.

11. The method of claim 3, wherein said stripes are more than 10 mm in width.

12. The method of claim 1, wherein said larger package contains 6, 8 or 10 said inner packages.

13. The method of claim 1, wherein said sanitary article is a feminine sanitary napkin.

14. The method of claim 1, wherein said sanitary article is a feminine sanitary pad.

15. The method of claim 1, wherein said sanitary article is a baby diaper.

16. The method of claim 1, wherein said sanitary article is a pantyliner.
